(19) United States
(12) Patent Application Publication
(10) Pub. No.: US 2022/0339225 A1
(43) Pub. Date: Oct. 27, 2022

(54) METHODS FOR REDUCTION OF BITTERNESS IN CANNABINOIDS USING MYCELIAL MATERIALS

(71) Applicant: MYCOTECHNOLOGY, INC., Aurora, CO (US)

(72) Inventors: James Patrick LANGAN, Aurora, CO (US); Bhupendra Kumar SONI, Aurora, CO (US); Anthony J. CLARK, Aurora, CO (US); Lisa SCHMIDT, Aurora, CO (US)

(73) Assignee: MYCOTECHNOLOGY, INC., Aurora, CO (US)

(21) Appl. No.: 17/642,328

(22) PCT Filed: Sep. 18, 2020
(86) PCT No.: PCT/US2020/051530

§ 371 Date: Mar. 11, 2022

Related U.S. Application Data
(60) Provisional application No. 62/903,544, filed on Sep. 20, 2019.
(60) Provisional application No. 62/925,085, filed on Oct. 23, 2019.

(51) Int. Cl. A61K 36/068; A61K 9/107; A61K 9/00; A61K 47/44

(52) U.S. Cl. CPC A61K 36/068; A61K 9/107; A61K 9/0053; A61K 47/44

(57) ABSTRACT

Provided is a composition for oral administration, which can include a combination of an extracellular portion from a mycelial aqueous culture comprising a filamentous fungus, and at least one of a cannabis oil or a cannabinoid and optionally, a material comprising a surfactant or an oil, and wherein the composition has a reduced undesirable taste compared with the same composition lacking the extracellular portion. Also provided is a method for reducing undesirable tastes in a composition comprising a cannabinoid or cannabis oil for oral administration, which include adding an extracellular portion from a mycelial aqueous culture comprising a filamentous fungus to the cannabinoid-containing or cannabis oil-containing product for oral administration in an amount sufficient to reduce at least one undesirable taste in the composition.

### CROSS-REFERENCE TO RELATED APPLICATIONS

This application claims priority to and the benefit of U.S. Provisional Application Ser. No. 62/903,544, filed Sep. 20, 2019 and 62/925,085, filed Oct. 23, 2019, which is incorporated herein by reference in its entirety.

### BACKGROUND

Cannabis oils and their individual cannabinoids, including Δ9-tetrahydrocannabinol (Δ9-THC), cannabidiol (CBD), and the like, and their formulations, such as oils, tinctures, inhalable forms, and edibles/drinks, are used to provide analgesia, help alleviate nausea and emesis, and have anti-spasmodic, anti-inflammatory, anti-convulsant, anti-psychotic, anti-oxidant, neuroprotective, anti-inflammatory, anti-cancer, and immunomodulatory effects. Given the therapeutic benefit, it would be advantageous to develop a composition in which cannabinoids are delivered orally or sublingually.

However, the taste associated with cannabinoids and cannabinoid oils makes providing products with acceptable organoleptic qualities difficult. Cannabinoids have a number of off-flavors, such as grassy, bitter, and other undesirable flavor notes. Full-spectrum cannabis extracts are known to have a bitter and earthy taste, although upon further extraction, the intensity of the taste may drop due to removal of some of the bitter components such as terpenes and flavonoids.

In their natural form, cannabis compounds known as cannabinoids are oils that, like other oily foods and edible oils, separate from water-based liquids. In order to incorporate cannabis products oils into food or beverages (which are typically water-based), it is known to create cannabis extract nanoemulsions (a.k.a., water-soluble CBD and THC). Infusing beverages with cannabinoid (e.g., CBD and/or THC)-containing translucent nanoemulsions (ideally, averaging ˜20 nanometers in droplet size diameter) is advantageous as nanoemulsions of this size do not change the original appearance of the beverages. Emulsions typically incorporate carrier oils, surfactants (emulsifiers), antioxidants and preservatives, using emulsifiers such as lecithin. However, creating a nanoemulsion can amplify bitter flavors due to the increase in the surface area exposed to the taste buds.

Previously, the inventors have filed patent applications to a bitter blocker obtained from mycelial materials, comprising an extracellular portion of an aqueous filamentous fungal culture. These patent applications U.S. patent application Ser. No. 15/144,164, filed May 2, 2016, U.S. patent application Ser. No. 14/836,830, filed Aug. 26, 2015, U.S. Provisional Application No. 62/042,071, filed Aug. 26, 2014, U.S. Provisional Application No. 62/253,567, filed Nov. 10, 2015, U.S. Provisional Application No. 62/281,546, filed Jan. 21, 2016, the disclosure of each of which is hereby incorporated by reference herein in its entirety.

What is desired is a way of manufacturing a cannabis in, for example, an oil, fat, and/or surfactant-containing product for oral administration, such as a beverage that provides reduced undesirable tastes that are associated with the cannabis. Thus, a need remains in the art for products having reduced levels of undesirable taste components and for methods of obtaining such products. The present invention is directed toward overcoming one or more of the problems discussed above.

### SUMMARY OF THE INVENTION

In an embodiment, the present invention includes a composition for oral administration, wherein the composition comprises a combination of an extracellular portion from a mycelial aqueous culture comprising a filamentous fungus, and at least one of a cannabis oil or a cannabinoid and optionally, a material comprising a surfactant or an oil, and wherein the composition has a reduced undesirable taste compared with the same composition lacking the extracellular portion. The filamentous fungus can be one or more of, or selected from the group consisting of: Ganoderma lucidum, Ganoderma applanatum, Cordyceps sinensis, Cordyceps militaris, Hericium erinaceus, Lentinula edodes, Agaricus blazei, Grifola frondosa, Auricularia auricula, Flammulina velutipes, Trametes versicolor, Morchella spp., Inonotus obliquus, Laricifomes officinalis, Fomes fomentarius, Fomes officinalis, Fomes fomitopsis, Tricholoma matsutake, Boletus edulis, Clitocybe nuda, Clitocybe saeva, Plearotus spp., Tremella fuciformis, Piptoporus betulinus, Polyporus umbellatus, Pholiota nameko, Volvariella volvacea, Hypsizygus marmoreus, Stropharia rugosoannulata, and Laetiporus sulphureus. The composition of claim 1, wherein the extracellular portion of the mycelial aqueous culture may be obtained by filtration or centrifugation of the mycelial aqueous culture.

The extracellular portion of the mycelial aqueous culture is prepared by a method which includes the steps of, optionally, culturing the mycelial aqueous culture in a media; separating the extracellular portion from the mycelial cells; and collecting the extracellular portion of the mycelial aqueous culture.

In embodiments, the reduced undesirable taste comprises at least one of reduced bitter tastes, reduced undesirable aftertastes, and/or reduced astringency compared to the product for oral administration alone. In embodiments, prior to incorporation into the inventive compositions, the extracellular portion from the mycelial aqueous culture can be pasteurized or sterilized and may be collected from the culture by filtration or centrifugation.

The compositions may be a beverage, a food, or a dietary supplement. Prior to incorporation into the composition, the cannabis oil or cannabinoid and/or the extracellular portion, either separately or together, may be incorporated into an oil-in-water or a water-in-oil emulsion. In embodiments, the composition may optionally comprise a surfactant and/or and oil; in embodiments, the surfactant is lecithin, such as soy lecithin, sunflower lecithin, or egg lecithin; in some embodiments, the oil is a medium chain triglyceride. The emulsion may then be incorporated into the product for oral administration.

In embodiments, the present invention includes a method for reducing undesirable tastes in a composition comprising a cannabinoid or cannabis oil for oral administration. The steps may include adding an extracellular portion from a mycelial aqueous culture comprising a filamentous fungus to the cannabinoid-containing or cannabis oil-containing product for oral administration in an amount sufficient to reduce at least one undesirable taste in the composition for oral administration.

### DETAILED DESCRIPTION OF THE INVENTION

The following detailed description illustrates a few embodiments in further detail to enable one of skill in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art, however, that other embodiments of the present invention may be practiced without some of these specific details. Several embodiments are described and claimed herein, and while various features are ascribed to different embodiments, it should be appreciated that the features described with respect to one embodiment may be incorporated with other embodiments as well. By the same token, however, no single feature or features of any described or claimed embodiment should be considered essential to every embodiment of the invention, as other embodiments of the invention may omit such features.

Unless otherwise indicated, all numbers used herein to express quantities, dimensions, and so forth used should be understood as being modified in all instances by the term “about.” In this application, the use of the singular includes the plural unless specifically stated otherwise, and use of the terms “and” and “or” means “and/or” unless otherwise indicated. Moreover, the use of the term “including,” as well as other forms, such as “includes” and “included,” should be considered non-exclusive. Also, terms such as “element” or “component” encompass both elements and components comprising one unit and elements and components that comprise more than one unit, unless specifically stated otherwise.

In one embodiment, the present invention is based on the discovery that fungi cultured media (or extracellular material) (on any media as described herein) such as Cordyceps sinensis-extracellular material or portion thereof, can be used directly as a flavorant, flavor enhancer, or flavor blocker. The cultured media can be dried, diluted, concentrated, or used neat in the forms of a concentrate, dried powder, and the like.

The inventors have found that the mycelial culture, in one embodiment, need only be filtered (with, e.g., cheesecloth, coffee filter, 0.2 micron filter) and pasteurized to isolate the extracellular portion (fluid), which is capable of providing taste-altering properties to a number of edible materials and products for oral administration, including those products containing cannabis oil, hemp oil, and/or cannabinoids.

In one embodiment, the present inventors have found that the a portion of a fungal aqueous culture fluid, the extracellular portion (containing reduced amounts of mycelium, herein also referred to as the “mycelium-free portion”) when added directly a composition for oral administration comprising a material such as, for example, a surfactant or an oil and cannabis or cannabinoids, has the ability to improve undesirable tastes in the composition, that are due to the surfactant or the oil in the composition. Such undesirable tastes include bitter tastes, grassy tastes, and/or other undesirable cannabis flavors. Flavor/taste improvement also includes reduction of characteristic tastes and/or aftertastes associated with cannabinoids and cannabis and hemp oils, including, without limitation, a bitter flavor, a grassy flavor, an herbal flavor, a barnyard flavor.

Improved flavor of products treated by products or compositions of the invention may be measured in a variety of ways, such as the chemical analysis which demonstrate improved sweetness, reduced bitterness and/or mitigated taste defects. Taste tests with taste panels may also be conducted to provide qualitative data with respect to improved taste(s) in the products, with the panels determining whether decreased taste defects have been exhibited in the treated products.

Sensory evaluation is a scientific discipline that analyses and measures human responses to the composition of food and drink, e.g. appearance, touch, odor, texture, temperature and taste. Measurements using people as the instruments are sometimes necessary. The food industry had the first need to develop this measurement tool as the sensory characteristics of flavor and texture were obvious attributes that cannot be measured easily by instruments. Selection of an appropriate method to determine the organoleptic qualities, e.g., flavor, of the instant invention can be determined by one of skill in the art, and includes, e.g., discrimination tests or difference tests, designed to measure the likelihood that two products are perceptibly different. Responses from the evaluators are tallied for correctness, and statistically analyzed to see if there are more correct than would be expected due to chance alone.

In the instant invention, it should be understood that there are any number of ways one of skill in the art could measure the sensory differences.

In an embodiment, the compositions of the invention, e.g., produced by methods of the invention, have reduced bitterness, as measured by sensory testing as known in the art. Such methods include change in taste threshold, change in bitterness intensity, and the like. At least 10% or more change (e.g., reduction in) bitterness is preferred. The increase in desirable flavors and/or tastes may be rated as an increase of 1 or more out of a scale of 5 (1 being no taste, 5 being a very strong taste.) Or, a reference may be defined as 5 on a 9 point scale, with reduced bitterness or at least one flavor as 1-4 and increased bitterness or at least one flavor as 6-9. The invention includes reduction in one or more of the named organoleptic qualities (bitter tastes, grassy tastes, and/or other undesirable surfactant or oil-related flavors) as discussed herein.

Additionally, the organoleptic qualities of the compositions of the invention may also be improved by processes of the current invention. For example, deflavoring can be achieved, resulting in a milder flavor and/or with the reduction of, for example, bitter and/or astringent tastes and/or other undesirable surfactant or oil-related flavors. The decrease in undesirable flavors and/or tastes as disclosed herein may be rated as a decrease of 1 or more out of a scale of 5 (1 being no taste, 5 being a very strong taste.)

Accordingly, the present invention relates to a composition for oral administration, wherein the composition comprises a combination of an extracellular portion from a mycelial aqueous culture comprising a filamentous fungus and a material comprising a cannabinoid or cannabis, optionally together with a surfactant or an oil, and wherein the composition has a reduced undesirable taste due to the surfactant or the oil in the composition, as well as methods by which to reducing undesirable tastes of a such a composition, by adding an extracellular portion from a mycelial aqueous culture comprising a filamentous fungus to the material comprising cannabis or cannabinoid and optionally, a surfactant or an oil in an amount sufficient to reduce at least one undesirable taste due to the cannabis or cannabinoid in the composition for oral administration. The compositions comprising the combinations have reduced undesirable taste or tastes relative to the products for oral administration alone. The compositions optionally comprise cannabis oil, hemp oil, and/or at least one cannabinoid, optionally, an oil or a surfactant in an emulsified format.

Specifically, the inventors used an extracellular portion of a C. sinensis aqueous culture to mix with a cannabinoid cannabis- or hemp-oil and optionally, oil or surfactant-containing composition, for the reason that the extracellular portion of an aqueous culture has taste improving and/or bitter blocker properties. The extracellular portion may be further purified, for example, to increase solubility, and may be dried, such as spray-drying, and combined with products for oral administration to improve the food products' taste profiles, including reducing bitter tastes and/or aftertastes. The present invention thus discloses a bitter blocker (the extracellular portion) that is effective for reducing undesirable flavors in cannabinoid or cannabis/hemp oil-containing compositions for oral administration.

In one embodiment, the present invention includes a method for reducing undesirable tastes of a product for oral administration, which includes the steps of culturing a mycelial aqueous culture in an aqueous media, collecting an extracellular portion of the culture, and adding the extracellular portion to the material to reduce its undesirable tastes. In an embodiment, the material is part of a composition or product for oral administration to enhance the food products' taste. In an embodiment, the product for oral administration comprises at least one cannabinoid and/or a hemp oil and/or a cannabis oil. In another embodiment, the methods include the step of adding an extracellular portion of a mycelial aqueous culture to a composition comprising cannabis oil, hemp oil and/or a cannabinoid in an amount effective to reduce at least one undesirable taste of the composition comprising cannabis oil, hemp oil and/or a cannabinoid.

In one embodiment, the mycelial aqueous culture is carried out in a bioreactor pressure vessel which is ideally constructed with a torispherical dome, cylindrical body, and spherical cap base, jacketed about the body, equipped with a magnetic drive mixer, and ports through curled-in jacket spaces to provide access for equipment comprising DO probes, pH meters, conductivity meters, thermocouples, etc., as is known in the art. These meters and probes should be data-logged. In one embodiment, the cylindrical base has a valve connected to a harvesting line which is teed off to a valve to another tee, which is teed-off to a floor sink and in-line with a CIP skid, the harvesting line tee in-line to a pasteurization skid, and finally a drying device, such as a spray dryer, fluid bed dryer, conical dryer, or other drying applications. In one embodiment, the processed mycelial aqueous culture can be packaged immediately from the dryer. A sample should be kept as control and an appropriate sample sent to a third-party quality control, Certificate of Analysis provider. Air can be provided by an air receiver tank connected to a 120/240 V air compressor. The air compressor releases air through a pressure regulator with upstream and downstream valves, immediately upstream of the upstream valve being a tee, teed-off to a valve leading to another tee, teed-off to a valve to a CIP skid, in-line with a valved steam supply, the post pressure regulator valve in-line to a valve and 0.2 μm stainless steel filter (which can be cleaned in a sonicating sink) in a stainless steel cartridge housing, which leads to an optional check valve to obligate valve on the dome of the pressure vessel, the final valve system optionally being upstream of the check valve, teed off to a y-piece which leads to two similar check valve to valve setups to 360° sprayballs. The two sprayballs are placed to account for the shadow presented by the air percolator that extends through the vessel. Pressure gauges along the set-up may be strategically placed to monitor pressure, and flow meters used to monitor air supply rates. Additional gas receiver tanks, such as oxygen tanks, can be placed in-line between the pressure regulator and the filters to calibrate partial pressures of any gas. The inventors recommend back to back filter cartridges, though this is not necessary. The gas is exhausted through a check valve with low-cracking pressure, such as a gate-valve, or a spring check valve with 2 to 3 psi cracking pressure, to a back-pressure regulator that holds the vessel at 5 to 25 psi. The back-pressure regulator can also lead to a steam trap and floor-sink. In one embodiment the set-up provides 0.5 to 5.0 ACH. Other engineering schemes known to those skilled in the art may also be used.

The reactor preferably is outfitted with a means for sterile inoculation. In one embodiment, to inoculate the reactor, a glycerol stock solution of fungi, consisting of a valved autoclavable (e.g. polypropylene) container, is taken out of the freezer, removed from its seal and attached to a cross, in-line with a valve to the chamber. The cross cross-line is valved on both ends, with the upstream valve connected to a stainless steel cartridge housing holding a stainless steel 0.2 μm filter. This line is connected to a valved tee (also valved on the upstream side) in-line to the main air supply line. Downstream of the cross is a valve to a steam strap to a floor-sink. The steam is run to sterilize the air between the glycerol stock and the valve to the chamber. Once sterilized and cooled, the vacuum between the glycerol stock and the valve to the chamber is broken. The valves on either side of the cross are closed, and the valves on the glycerol stock and pressure vessel are opened to inoculate the media. Other engineering schemes known to those skilled in the art may also be used.

The reactor should be outfitted to be filled with water. The water supply system is ideally a WFI system, with a sterilizable line between the still and the reactor. Solid media ingredients should be added to the tank pre-sterilization, ideally through a vacuum conveyor system. High temperature sterilizations are fast enough to be not detrimental to the media. Once the water is added, the tank should be mildly agitated and inoculated. In another embodiment, solid media ingredients are added to filtered or distilled water and the liquid media is sterilized at high temperatures and pumped through a sterile line into the pressure vessel. In another embodiment, the tank is filled with filtered or distilled water, the solid media ingredients are added, and the media is sterilized by steaming the either the jacket, chamber, or both, while the media is optionally being agitated.

At least one scale-up reactor should be used before approaching tanks with volumes on the order of 1×10⁵. As many as 3 to 4 are recommended. The inventors recommend going from the order of 1×10⁰ L to 1×10² L to 1×10⁴ L to 1×10⁵⁻⁶ L. Richer media can be used for the scale-up reactors and pre-glycerol stock culturing motifs.

The glycerol stock disclosed herein is prepared, in one embodiment, by a simple propagation motif of Petri plate to 0.1 L to 4 L Erlenmeyer shake flask to 50% glycerol stock. Petri plates can comprise agar in 25 to 35 g/L in addition to variations of the media described above for bioreactor motif. Conducted in sterile operation, chosen Petri plates growing anywhere from 3 to 90 days can be propagated into 4 L Erlenmeyer flasks (or 250 to 1,000 mL Wheaton jars) for incubation on a shaker table. The smaller the container, the faster the shaker should be. The inventors recommend anywhere from 40 to 160 RPM depending on container size, with about a 1″ swing radius. After shaking for 1 to 10 days, an aliquot (e.g. 10 to 500 mL) of the shake flask can be poured into a sterile, valved autoclavable container, which is then adjusted with sterile, room temperature glycerol to 40 to 60% (v/v). The glycerol stocks can be sealed with a water tight seal and can be placed into a sterile plastic bag, sealed, and placed into the freezer at −20° C. for storage and eventual cold shipping to any manufacturing site. The freezer is ideally a constant temperature freezer. Aqueous culture stocks not adjusted to glycerol may also be used and stored at 4° C. or −20° F. Glycerol stocks stored at 4° C. may also be used.

The present invention makes use of the production of edible liquid mycelial culture, as is known in the art and also disclosed elsewhere, e.g., PCT/US14/29989, filed Mar. 15, 2014, PCT/US14/29998, filed Mar. 15, 2014, U.S. 61/953,821, filed Mar. 15, 2014, U.S. 61/953,823, filed Mar. 15, 2014, U.S. 62/042,071, filed Aug. 26, 2014, all of which are incorporated by reference herein in their entireties. Preferably, a nitrogen salt, if used, is ammonium acetate, as it is the most ‘natural’ salt. Other supplemental media ingredients include brown rice syrup, molasses, fruit purees (apple, etc.) in concentrations on the order of 1×10⁻² to 1×10² mL/L (or simply as the media), short grain brown rice flour, nutritional yeast flakes, carboxymethyl cellulose, carboxymethyl cellulose salts, whey, casein, and plant and seed protein. Ingredients are chosen so as to minimize possibilities for allergic reactions and provide high yield. Ammonium acetate is optionally incorporated as a batch fed ingredient.

In one embodiment, a fungus strain useful for the fungal component of the present invention in one embodiment is C. sinensis strain WC859, commercially available from Pennsylvania State University (The Pennsylvania State University Mushroom Culture Collection, available from the College of Agriculture Sciences, Department of Plant Pathology and Environmental Microbiology, 117 Buckhout Laboratory, The Pennsylvania State University, University Park, Pa., USA 16802). Fungal components useful in the present invention may be prepared by methods described herein. Other methods known in the art may be used.

Alternatively, the fungal aqueous culture can include other species of fungi from genus Cordyceps, Ophiocordyceps, Elaphocordyceps, Metacordyceps, such as, for example, C. militaris. Many other species exist in the genus, however, these species are generally not cultivated commercially. However, it is expected that, for example, C. scarabaeicola, C. takaomontana, Ophiocordyceps dipterigena, Ophiocordyceps amazonica, C. cylindrica, Cordyceps sphecocephala, Metacordyceps martialis, Ophiocordyceps melonlonthae, Ophiocordyceps nutans, Ophiocordyceps curculionium, Ophiocordyceps australis, Ophiocordyceps tiputini, Cordyceps caloceroides, and Cordyceps variabilis will have the same or similar bitter blocking ability as C. sinensis.

Alternatively, fungi suitable for the present invention comprises: Ganoderma lucidum, Ganoderma applanatum, C. militaris, Hericium erinaceus, Lentinula edodes, Agaricus blazei, Grifola frondosa, Auricularia auricula, Flammulina velutipes, Trametes versicolor, Morchella spp., Inonotus obliquus, Laricifomes officinalis, Fomes fomentarius, Fomes officinalis, Fomes fomitopisis, Tricholoma matsutake, Boletus edulis, Clitocybe nuda, Clitocybe saeva, Plearotus spp., Tremella fuciformis, Piptoporus betulinis, Polyporus umbellatus, Pholiota nameko, Volvariella volvacea, Hypsizygus marmoreus, Stropharia rugosoannulata, Laetiporus sulfureus, and combinations thereof.

In one embodiment, the invention includes a method for preparing a mycelium-free or extracellular portion of the mycelial aqueous culture after culturing. The mycelium-free portion or extracellular portion includes mycelial biomolecular extracellular portion solids, cellular material and residual media of the mycelial aqueous culture.

As disclosed hereinabove, to prepare the culture, the prepared media is inoculated into a container of sterilized human grade media in water preferably filtered through any method known in the art, such as reverse osmosis, deionization or distillation. In another embodiment the water is not filtered. In another embodiment the media is animal grade. As disclosed, the flask and media can be sterilized by any method known in the art, such as in situ exposure to 250° F. at 23 PSI saturated steam for an appropriate amount of time, such as 2-2.5 hour for a 4.0 L Erlenmeyer flask filled with 1.5 L of media. The sterilized flask can be inoculated once cool by any means known in the art, such as by a Petri plate, floating or submerged liquid culture, myceliated agricultural material, glycerol stock, etc. The flask is ready for use after 3-60 days of appropriate culturing as is known in the art, such as on a shaker table at 130 RPM at room temperature in a cleanroom. A control Petri plate of the residual culture left in the flask can be made to ensure the flask is void of contamination. The flask can also be used to scale into a larger bioreactor (e.g. 5-500 L) made of the same quality media, which can be used in similar manner.

In some embodiments, the fungal aqueous culture is C. sinensis grown in an aqueous culture media consisting of 8 g/L organic potato starch powder and 0.8 g/L organic carrot powder. This minimal medium has been found by the inventors to be an effective media recipe for producing the bitter blocker (taste enhancement food product) as previously described. The bitter blocking effect/taste enhancement of the product of the invention can be lost with different media, such as the addition of 20 g/L organic fruit puree, which can introduce flavor defects. The resulting extracellular portion powder may be used as a bitter blocker in product applications as discussed herein.

After a suitable time for culturing, which can be determined by one of skill in the art, the mycelium-free or extracellular portion (as defined herein) can be collected from the culture. This mycelium-free portion of the liquid mycelial aqueous culture may optionally be used to improve and/or enhance the taste of a food product. Culturing can take place, for example, for between about one and about sixty days, between about two and about fifty days, between about three and about forty days, between about four and about thirty days, between about five and about twenty-five days, between about six and about twenty days, between about seven and about fifteen days, between about eight and about twelve days, and between about nine and about ten days. The length of time for culturing can be determined by, for example, economic considerations for number of days in culture and the degree of taste enhancement observed for a particular culture time.

The culture to use in the present invention may be any aqueous culture comprising mycelium, for example, submerged or floating culture. A submerged culture is generally agitated, whereas the floating culture is minimally agitated, which allows the mycelia to grow in a mat-like form. The portions of the culture to use with the present invention includes any and all parts or portions of the culture, including mycelium, culture extracellular portion or filtrate, or any proportions or fractions thereof. In one embodiment, the culture may be blended (mechanically or otherwise) prior to use, and the entire blended material used, or some fraction thereof. In some embodiments, the portion of the culture to use is the portion of the culture which is commonly understood as the “cell culture extracellular portion” or “cell culture filtrate”, i.e., the fluid portion of the culture which has been separated from the mycelial cells, and contains a relatively smaller or lesser amount of mycelium as opposed to a mycelial cell portion, which is enriched in mycelial cells, but will still contain some fluid portion. Thus, it should be understood that this fluid tissue culture extracellular portion will also commonly contain mycelia, even if not visible to the eye or even easily visible under a microscope. This portion of the culture is called herein the “mycelial-free” portion for convenience, however, as stated it should be understood that this portion will commonly contain some minimal amount of mycelia, even if not visible to the eye.

In order to prepare the mycelium-free portion of the culture, the mycelium can be removed by any method known in the art to separate cell culture extracellular portion fluids. For example, the culture may be filtered by any means known in the art to obtain the filtrate, such as, for example, 0.2 μm filters and the like. Alternatively, the mycelium-free portion of the culture may be collected by centrifugation. The collected mycelium-free portion of the cultured mycelial aqueous culture may be referred to herein as collected extracellular portion, extracellular portion, extracellular portion fluid, extracellular portion, filtrate, product, and similar terms such as the taste-enhancing product or bitter blocker/blocking product, or bitter blocker.

The extracellular portion may comprise fractions thereof of the extracellular portion. The portions of the culture to use with the present invention includes any and all parts or portions of the culture, including mycelium, culture supernatant or filtrate, or any proportions or fractions thereof. In one embodiment, the culture may be blended (mechanically or otherwise) prior to use, and the entire blended material used, or some fraction thereof. In some embodiments, the portion of the culture to use is the portion of the culture which is commonly understood as the “cell culture supernatant” or “cell culture filtrate”, i.e., the fluid portion of the culture which has been separated from the mycelial cells, and contains a relatively smaller or lesser amount of mycelium as opposed to a mycelial cell portion, which is enriched in mycelial cells, but will still contain some fluid portion. Thus, it should be understood that this aqueous culture supernatant will also commonly contain mycelia, even if not visible to the eye or even easily visible under a microscope. This portion of the culture is called herein the “mycelial-free” portion for convenience, however, as stated it should be understood that this portion will commonly contain some minimal amount of mycelia, even if not visible to the eye.

In one embodiment, one or more of fractions of the cell culture filtrate or supernatant may be used. The fractions may be generated in any number of ways as known in the art, for example, by filtration using specific size cutoffs, size exclusion chromatography, dialysis using differently sized membranes, and the like. The size cutoffs can include, for example, a fraction of the material having an average size of less than 1 kD, less than 5K, less than 10 kD, less than 20 kD, less than 30 kD, less than 50 kD, less than 100 kD, less than 200 kD less than 400 kD, less than 800 kD. Alternatively, the embodiments can include a size fraction of greater than 1 kD, greater than 5K, greater than 10 kD, greater than 20 kD, greater than 30 kD, greater than 50 kD, greater than 100 kD, greater than 200 kD, greater than 400 kD, all named fractions in this section having an average molecular weight of less than 800 kD. Alternatively, the fractions can include a fraction of less than 1 kD, a fraction of between 1 kD and 5 kD, a fraction of between 1 kD and 10 kD, a fraction of less than 10 kD, or a fraction of greater than 10 kD. A fraction may be used on its own, or in combination with any other fractions.

In one embodiment, the one or more of the fraction of the filtrate or supernatant may have at least 1% of, at least 2% of, at least 3% of, at least 4% of, at least 5% of, at least 6% of, at least 7% of, at least 8% of, at least 9% of, at least 10% of, at least 12% of, at least 14% of, at least 16% of, at least 18% of, at least 20% of, at least 22% of, at least 24% of, at least 26% of, at least 28% of, at least 30% of, at least 35% of, at least 40% of, at least 45% of, at least 50% of, at least 55% of, at least 60% of, at least 65%, at least 70% of, at least 75% of, at least 80% of, at least 85% of at least 90% of the activity of the filtrate or supernatant.

In other embodiments, the composition can comprise, consist of, or consist essentially of one or more of the fractions identified above. In other words, the composition can comprise, consist of, or consist essentially of one of the fractions; any two of the fractions; any three of the fractions; any four of the fractions; any five of the fractions; any six of the fractions; any seven of the fractions, or more. Each fraction can include one or more of the individually named components and/or compounds as disclosed herein within that fraction. For example, the composition can comprise a fraction which includes one or more of an α-linked polysaccharide; an organic acid or salt thereof; a phenolic compound; a carbohydrate, ester, or ketone; a nucleoside or nucleotide; or a fatty acid, and any other combination thereof which includes one, two, three, four, five or six of the components. In one embodiment, the composition comprises a fraction of greater than 10 kD, greater than 15 kD, greater than 20 kD, greater than 40 kD, greater than 60 kD, greater than 80 kD, greater than 100 kD, greater than 150 kD, greater than 200 kD, greater than 300 kD, greater than 500 kD, all named sizes being less than about 800 kD.

Optionally, the aqueous culture can be treated to reduce or eliminate the viability of live organisms, such as pasteurization or sterilization, by methods known in the art. The collected aqueous culture may be pasteurized or sterilized either before or after separation to obtain the mycelium-free portion of the culture, by any method known in the art. In one embodiment the material is sterilized under conditions such as approximately 30 to 50 minute exposure to 250° F. saturated steam at 23 psi. Alternatively, the material can be pasteurized by holding the material in a hot water bath at 160 to 170° F. for 20 minutes, twice, cooling it back to room temperature in between runs.

The filtrate (collected extracellular portion) e.g., mycelium-free portion of a mycelial aqueous culture may have its volume or liquid component adjusted as determined by one of skill in the art to produce concentrates, diluates, or dried powders. In one embodiment, the filtrate may be optionally dried by any method known in the art, including the use of open-air drying, small batch desiccators, vacuform dryers, fluid beds or spray dryers, or freeze-driers to dry the liquid to a powder. The filtrate is, in one embodiment, dried following sterilization/pasteurization.

In one embodiment, the extracellular portion, as indicated herein, can optionally be prepared e.g., by spray drying a solution or suspension in carriers such as maltodextrin, starches, gum arabic, or other acceptable carriers or fillers known in the art. Spray drying is a well-known operation often employed by the flavor industry to render liquid flavors into a dry free-flowing powder form. It is a cost effective and efficient process to achieve this objective. It is customary to add certain emulsifiers as part of the formulation if the non-flavoring ingredient lacks or is deficient in emulsifying properties. Conventional spray-drying techniques are perfectly well documented in the prior art. See for example Spray-Drying Handbook, 4th ed., K. Masters, (1985) or other reference books on the subject-matter. Alternatively, the extracellular portion may be dissolved or suspended in a food grade alkylene glycol such as glycerol, or food grade alcohol.

The resulting extracellular portion product may be used to enhance the taste of a composition for oral administration comprising a cannabinoid, cannabis oil or hemp oil, optionally containing a surfactant, and may be mixed into the material or composition as described herein at concentrations of 0.1-1,000 ppm (mg per liter) and even higher depending on the nature of the application Determination of the amount of the taste enhancement product to use may be determined by one of skill in the art by trial with the goal to reduce or eliminate undesirable taste component in the food product and/or enhance the food product's taste, without introducing flavor defects.

A general range of concentrations of extracellular portion (bitter blocker) as a dried powder to use with various food products is provided herein. It is within the skill in the art to determine optimum ratios of the extracellular portion to use with a particular product, based on taste profiles. For example, at too high concentrations of extracellular portion, the flavor enhancing effect will cease to be or the product will introduce flavor defects into the final material. At too low of a concentration of extracellular portion, there will be an insufficient degree of taste improvement. The concentration of the agricultural material ultimately determines the ideal bitter blocker concentration. For example, serial dilution/concentration can be used as a tool in determining the upper and lower threshold concentrations use of the extracellular portion. Formulate the bitter blocker into the material at whatever initial desired concentration one wants to test. If it provides the desired flavor change, halve the concentration until the flavor change is insufficient. Take the final concentrations between what worked and what did not, and apply the bitter blocker at the average. If it works, halve the concentration until it no longer works, and the concentration above the one that doesn't work is the lower threshold concentration. If it doesn't work, double the concentration until it does. The lower threshold concentration can be doubled indefinitely to reach the upper threshold concentration, wherein the taster determines whether the flavor modifying effect is eventually lost or the bitter blocker starts to introduce a flavor defect.

The powder may also be rehydrated, filtered and re-dried to increase solubility of the product. The spray dried product has high solubility and optionally is not rehydrated before use, and may be simply mixed in as a powder with a food product (particularly in non-nutritive sweetener applications). Alternatively, the taste enhancement food product may be combined with a food product in liquid form, and optionally the food product/taste enhancement product may be dried together. The extracellular portion powder may also be dried in a fluid bed, or spray dried onto a fluidized product and even agglomerated, such as in the production of a steviol glycoside mixture comprising the product.

Accordingly, the present invention includes a method to make a composition for oral administration with at least one reduced undesirable taste, or a method to reduce an undesirable taste of a material comprising cannabis, a cannabinoid, or hemp oil, in a composition for oral administration, comprising mixing an extracellular portion of a mycelial aqueous culture with the material, in an amount effective to reduce the at least one undesirable taste due the cannabis oil, hemp oil, or a cannabinoid.

Accordingly, the present invention includes a method to make a composition for oral administration with at least one reduced undesirable taste, or a method to reduce an undesirable taste of a material comprising a cannabis oil, hemp oil, or a cannabinoid in a composition for oral administration, comprising culturing a mycelial aqueous culture in a media, collecting the extracellular portion, and combining the extracellular portion of the culture and mixing it with the material or composition. Appropriate fungi to use, appropriate media, appropriate methods of collecting the mycelium free portion of the extracellular portion are disclosed herein. The extracellular portion may be optionally concentrated, diluted or dried as disclosed herein, and may be combined with any food product as disclosed herein prior to use. The present invention also includes combination products comprising one or more food product(s) and an extracellular portion made from a mycelial aqueous culture made by the processes disclosed herein.

Therefore, in another embodiment, provided is a composition comprising a combination of a material comprising cannabis oil, hemp oil, at least one cannabinoid and optionally, a surfactant, and extracellular portion from a mycelial aqueous culture. In one embodiment, the mycelial aqueous culture is produced by methods of the present invention.

In one embodiment, the mycelium-free portion from a mycelial aqueous culture is a dried or partially dried filtrate or extracellular portion from the mycelial aqueous culture. The composition may include the mycelium-free portion of a mycelial aqueous culture obtained from a fungus as previously defined herein, and may include, for example, Cordyceps sinensis, and/or Cordyceps militaris.

The mycelium free portion of the mycelial aqueous culture may be obtained by any methods known in the art, including methods disclosed herein. Such methods include the steps of culturing a mycelial aqueous culture in a media, separating the mycelium-free fluid from the mycelial cells, and collecting the mycelium-free fluid as the mycelium-free portion of the mycelial aqueous culture.

The composition, in some embodiments, has a taste enhancement which includes reduced bitter tastes, reduced undesirable aftertastes, and/or reduced grassy/herbal tastes, compared to the food product alone.

Compositions may be formed from food products that are dried prior to combination with the mycelium-free portion of a mycelial aqueous culture. In some embodiments, prior to combination with a food product, the mycelium-free portion of a mycelial aqueous culture is dried. Thus, a dried food product may be combined with a dried mycelium-free portion of a mycelial aqueous culture to form the composition.

As defined herein, the present invention may include at least one cannabinoid, cannabis oil, hemp oil, and/or combinations thereof. The cannabis plant has many naturally occurring substances that are of great interest in science and medicine. Isolated compounds from the cannabis plant include Δ9-tetrahydrocannabinol (THC), cannabidiol (CBD), cannabichromene (CBC), cannabigerol (CBG), cannabidivarin (CBDV), among other compounds. While THC has psychoactive effects, CBD, CBC, CBG, and CBDV do not. Isolated compounds from the cannabis plant are called cannabinoids. These are among the most prominent compounds in the family of compounds extracted from the cannabis plant referred to as cannabinoids. There are a total of at least eighty-five (85) cannabinoids isolated from the cannabis plant. Many researchers have confirmed the medicinal value of cannabinoids. Cannabinoids have been investigated for possible treatment of seizures, nausea, vomiting, lack of appetite, pain, arthritis, inflammation, and other conditions.

Cannabinoids can be isolated by extraction from cannabis plants. Plants in the cannabis genus include Cannabis sativa, Cannabis ruderalis, and Cannabis indica. These plants are the natural sources of cannabinoids. Cannabinoids are also available in synthetic forms.

Cannabis oil or hemp oil may be obtained by cold pressing and/or extracting the entire Cannabis sativa L. plant or hemp plant. Depending on the specific cultivar, the resulting cannabis oil may contain certain percentages of THC and CBD, as well as other cannabinoids. Other impurities such as linoleic acid and a-linoleic acid, β-caryo phyllene, myrcene, and β-sitosterol may also be present.

Hemp oil or cannabis oil generally contain Δ9-tetrahydrocannabinol, cannabidiol, cannabigerol, among other cannabinoids. Specific weight percentage of each cannabinoid varies, depending on naturally occurring amount and processing methods.

Alternatively, hemp oil or cannabis oil may be processed to remove the fatty acid content, leaving concentrated or crystalized cannabinoids in solid form.

Cannabinoids, cannabis and hemp oil according to the present invention therefore can comprise naturally occurring molecules that are purified from hemp, cannabis, or another plant source. The cannabinoid, hemp oil, cannabis oil, or combination thereof can be synthetically prepared. The cannabinoid, hemp oil, cannabis oil, or combination thereof can be derived from genetically engineered microorganisms grown in a controlled environment. The cannabinoid, hemp oil, cannabis oil, or combination thereof can be derived from a genetically engineered plant that is grown either in a field or in a greenhouse. The cannabinoid, hemp oil, cannabis oil, or combination thereof can be derived from plant cell cultures. The cannabinoid, hemp oil, cannabis oil, or combination thereof can comprise hemp oil, cannabis oil, and cannabinoids homogenized together, for example.

A cannabis or hemp oil is produced by solvent extraction, e.g. ultrasonic extraction or ultrasonically-assisted supercritical CO₂ extraction of cannabis or hemp. For the production of hash oil, preferably dry, decarboxylated plant material is used. The following liquids are frequently used solvents for hash oil extraction: ethanol, chloroform, dichloromethane, petroleum ether, naphtha, benzene, butane, methanol, isopropanol, and olive oil. There are at least 113 identified types of cannabinoids in cannabis. Cannabinoids are active phytochemical substances with psychoactive and/or medical effects. THC or tetrahydrocannabinol is the primary psychoactive compound in cannabis. CBD or cannabidiol is another important ingredient with therapeutic effects. Cannabinoids are lipophilic and potentially acid-labile compounds. Cannabinoids are insoluble in water, but they are very soluble in non-polar solvents (e.g. fat and oil). To improve the digestion of cannabinoids, mix cannabis extracts into carrier oils.

As defined herein, the present invention may further include a material or composition comprising at least one oil and/or surfactant. Preferably, the oil and/or surfactant are edible or food-grade. Surfactants generally are defined as compounds that lower the surface tension (or interfacial tension) between two liquids, between a gas and a liquid, or between a liquid and a solid. Surfactants may act as detergents, wetting agents, emulsifiers, foaming agents, and dispersants. Surfactants appropriate to use with the present invention include naturally occurring surfactants such a lecithin and proteins, such as those from milk, as well as polar lipids as monoglycerides. Other surfactants include synthetic surfactants such sorbitan esters and their ethoxylates and sucrose esters. Other emulsifiers that can be used in the invention include any food-grade emulsifier, including gum arabic or starch-based emulsifiers, as well as products such as ALKEST TW 80, SCATTICS, CANARCEL, POEGASORB 80, MONTANOX 80, TWEEN 80 LECITHIN: LIPOID.

In one embodiment, the emulsifier is lecithin, which is available in powder, granules and liquid form, and is suitable for food and beverage products. Lecithin is a generic term to designate any group of yellow-brownish fatty substances occurring in animal and plant tissues which are amphiphilic—they attract both water and fatty substances (and so are both hydrophilic and lipophilic), and are used for smoothing food textures, emulsifying, homogenizing liquid mixtures, and repelling sticking materials. Lecithins are mixtures of glycerophospholipids including phosphatidylcholine, phosphatidylethanolamine, phosphatidylinositol, phosphatidylserine, and phosphatidic acid.

The lecithin for use with the present invention includes an edible lecithin from any number of sources, but commercially the most common lecithins are soy lecithin, egg yolk lecithin, and sunflower lecithin. Lecithins may be further processed or treated prior to use. For example, lecithin is available in liquid or powder (spray dried) forms. Lecithins may be treated by filtration, chemical or enzymatic modification and de-oiling to produce purified or modified lecithins. The molecular structure and polar head groups may be modified by either enzymatic or chemical means.

Oils to use with the invention include edible oils. Oils include, without limitation, any edible oil from any plant, fungal, or animal source. In some embodiments, the oils include olive oil, coconut oil, corn oil, cottonseed oil, palm oil, peanut oil, rapeseed/canola oil, safflower oil, sesame oil, soybean oil, sunflower oil, and/or combinations thereof. Oils of the invention also include nut oils, such as almond oil, beech nut oil, brazil nut oil, cashew oil, hazelnut oil, macadamia nut oil, mongongo nut oil, pecan oil, pine nut oil, pistachio oil, walnut oil, pumpkin see oil or citrus oils, such as lemon oil, orange oil, grapefruit seed oil. Other oils include the “nutraceutical” type oils such as borage seed oil, blackcurrant seed oil, evening primrose oil, acai oil, black seed oil, flaxseed oil, amaranth oil, apricot oil, apple seed oil, argan oil, avocado oil, babassu oil, ben oil, borneo tallow nut oil, cape chestnut oil, carob pod oil, cocoa butter, cohune oil, coriander seed oil, date seed oil, grape see oil, hemp oil, and the like.

Oils are highly lipophilic, and are best mixed with lipids or fats instead of water. However, oils and fats are difficult to intermix with materials such as foods or beverages, which are water-based. Therefore, in one embodiment, the cannabis oils or cannabinoids of the invention are first prepared as an emulsion using a surfactant using known techniques, which is then diluted to form the composition for oral administration.

In one embodiment, the cannabis oil, hemp oil, or cannabinoid emulsions are suspensions of micron-size or nano-size cannabinoids and/or oil-containing products. The methods require intense shear to disperse the cannabinoid in water-based formulations. Cannabinoids are not freely soluble in water, but they show a good solubility in non-polar solvents such as oils and fats. It requires a suitable emulsification technique to overcome the immiscibility of cannabinoids e.g., oils in water. In embodiments, a cannabinoid or cannabis can be prepared as an oil-in-water emulsion or water-in-oil emulsion using known art techniques; any of the below emulsion techniques can be used with the present invention.

Oil-in-water emulsions are visually homogeneous mixtures that combine the properties of both oil and water, wherein water is the continuous phase and oil is the dispersed phase, stabilized by at least one surfactant (emulsifier). There are three main types of oil-in-water emulsions: macroemulsions, microemulsions and nanoemulsions. Any of these types of emulsions are suitable to use with the present invention.

Macroemulsions are common emulsions with droplets averaging over 1 micron in diameter and generally having wide droplet size distributions. They are unstable and tend to separate into an oil layer at the top and a water layer at the bottom, sometimes with a mixed layer in between. Due to their lack of stability, macroemulsions are generally not suitable as water-compatible cannabinoid delivery vehicles.

Microemulsions are translucent and thermodynamically stable mixtures of oil, water and surfactants having average droplet diameters below 100 nm. They can be spontaneously formed by “solubilizing” the oil in water or a beverage by very high amounts of surfactants—substantially higher than the amount of the oil. Because of many undesirable side-effects caused by concentrated surfactants (health issues, taste deterioration, etc.), the use of microemulsion-based, water-compatible cannabis oil formulations is disadvantageous. Furthermore, it is frequently impossible to achieve the desired cannabinoid concentration in a beverage without exceeding maximum permitted surfactant levels.

A typical microemulsion precursor (before the addition to a beverage) formulation is presented below:

Active ingredient (e.g., cannabis oil extract) 10.00%

Carrier oil (e.g., Miglyol 812) 30.00%

Surfactant 1 (e.g., oleic acid) 15.00%

Surfactant 2 (e.g., Tween 80) 33.75%

Surfactant 3 (e.g., Cremophor RH 40) 11.25%

For a typical cannabinoid dose of 10-15 mg, about 20-30 mg of cannabis oil extract (assuming 50-70% cannabinoid concentration) needs to be mixed into a beverage. With the microemulsion-type formulation, the same beverage will also end up with about 120-180 mg of surfactants, making it taste soapy and (for many surfactants) bitter, as well as potentially leading to regulatory compliance issues.

Liposomes are spherical structures with diameters from about 50 to 5000 nm formed by one or more concentric phospholipid bilayers with an aqueous phase inside and in-between the bilayers. Liposomes can entrap water-soluble (hydrophilic) active ingredients in their internal water compartment and water-insoluble (hydrophobic) active ingredients in their bilayer membrane. The latter property makes it possible to use liposomes to formulate water-compatible cannabis extracts. This, however, involves complex preparation procedures and, as with microemulsions, very high surfactant (lecithin phospholipids) concentrations. In addition, it is difficult to produce liposomal formulations using natural cannabis extracts, requiring the use of synthetic or isolated cannabinoids instead.

A typical liposome precursor (before the addition to a beverage) formulation is presented below:

Active ingredient (e.g., synthetic cannabinoids)—18.90%

Surfactants (e.g., lecithin phospholipids)—75.60%

Encapsulant (e.g., sodium alginate)—5.50%

Nanoemulsions are emulsions with narrow droplet size distributions centered below approximately 250 nm, or between 20 and 500 nm. A nanoemulsion is a thermodynamically stable isotropic system, which consists in two immiscible liquids (e.g. oil and water). One of the two immiscible liquids is dispersed very finely into the second phase in order to form a single phase. The addition of emulsifiers, e.g. surfactants and co-surfactants stabilize the emulsions by preventing the coalescence of the droplets. The size and shape of particles/droplets dispersed in the continuous phase defines the main difference between an emulsion and a nanoemulsion. Ostwald ripening is the major destabilization mechanism in THC or CBD nanoemulsions. Ostwald ripening is a diffusion degradation process, which means that an inhomogeneous structure such as an emulsion changes over time. The emulsion droplets increase over time as the smaller droplets dissolve and redeposit their material onto the larger droplets. The oil type has major influence on the intensity and rate of Ostwald ripening. This means that the choice of a suitable oil can affect the emulsion stability significantly. Nanoemulsions show an improved delivery of active ingredients (e.g. drugs).

Nanoemulsion-based formulations in which all droplets are smaller than 100 nm are optically translucent, achieving progressively higher degree of clarity as the droplet sizes are diminished. These formulations have several attractive properties, including low viscosity, high interfacial surface area and long-term kinetic stability. Nanoemulsions are made using significantly (about 10 times) lower surfactant amounts than microemulsions or liposomes. They are completely water-compatible and can be easily mixed into water or any beverage.

A typical nanoemulsion concentrate (with some water, but before the addition to a beverage) formulation is presented below:

Active ingredient (e.g., cannabis oil extract), 5.40%

Carrier Oil (e.g. olive oil), 7.20%.

Surfactant (e.g., Quillaja saponin) 2.00%

Water 85.4%

For a typical cannabinoid dose of 10-15 mg, requiring 20 to 30 mg of cannabis oil extract to be present in a beverage, a nanoemulsion-type formulation will only contain about 7-11 mg of surfactant, helping it retain its original taste. In addition, nanoemulsions can be produced using natural surfactants (formulation shown above), which avoids having any synthetic ingredients in the resulting products.

The reason that cannabis oil nanoemulsions require much lower surfactant concentrations than the alternatives is that the driving force for their formation is mainly mechanical instead of chemical. Nanoemulsions can be produced by utilizing ultrasonic cavitation-derived high shear forces, able to break the oil droplets down to nanometer sizes.

Laboratory, bench and industrial-scale ultrasonic liquid processors specially designed for the production of high-quality nanoemulsions are available.

In one embodiment, oils such as olive oil, coconut oil or MCT oil are used, which are known to assist the absorption of the active substances of cannabis into the digestive system. In a particular embodiment, to achieve an optimal absorption, cannabis extracts are mostly emulsified into oil and then further processed into an emulsion. For cannabis oil emulsions, lecithin is one of the most common emulsifiers. In a particular embodiment, a cannabis emulsion is prepared via sonication. For 2 wt % CDB nanoemulsion (oil in water), for example, a mixture of 2 wt % CBD oil, 2 wt % Polysorbate 80, 1 wt % Lecithin, 95 wt % water is used. For 5 wt % CDB nanoemulsion (oil in water) use: 5 wt % CBD oil, 3.3 wt % Polysorbate 80, 1.7 wt % Lecithin, 90 wt % water. Sonication may be carried out e.g., using ultrasonic equipment; in one embodiment, the ultrasonic energy used can be 300 to 400 Ws/g of sonication energy at amplitudes of 40 to 70 micron.

In one embodiment, the extracellular portion can be added to the composition for oral administration as part of an emulsion. The extracellular portion of the invention can be added to the emulsion at any number of different steps. One of skill in the art may determine the optimum method for incorporating the extracellular portion for maximum efficacy in a given application. In one embodiment, the extracellular portion is added to the water or aqueous portion of the emulsion prior to mixing with one or more other components of the emulsion. The extracellular component may be added to the water portion of the emulsion prior to incorporating into the other components of the emulsion. In order to obtain dissolution/suspension of the extracellular component, optionally, after addition, the aqueous portion is heated and/or sonicated, for example. Any method known in the art capable of enhancing dissolution/suspension may be used.

Optionally, the emulsifying agent may be added to the water portion before, after, or together with the extracellular component.

After the addition of the extracellular portion and/or emulsifying agent, the aqueous portion is then mixed with the optional oil portion of the emulsion. If the emulsifying agent was not added at a prior step, it may be added to an oil portion before, after or during the aqueous portion is mixed with the oil portion. The oil portion optionally contains the cannabinoid, cannabis oil, or hemp oil component, resulting in an emulsion that comprises the extracellular portion, oil, cannabinoids, and emulsifying agent. In an embodiment, a source of energy is applied to form the emulsion. In one embodiment, the emulsion is a nanoemulsion and is formed by methods known in the art for forming a nanoemulsion, including ultrasonication. Methods for forming a nanoemulsion are disclosed herein and known in the art.

Alternatively, the emulsion or nanoemulsion may be formed or admixed with the extracellular portion in the absence of cannabinoids, cannabis oil, or hemp oil, and later mixed or then blended with the cannabinoid, cannabis oil, or hemp oil to form the emulsion. The cannabinoid, cannabis oil or hemp oil may be in the form of an emulsion such as a nanoemulsion prior to blending with the extracellular portion emulsion or nanoemulsion.

The emulsion and/or nanoemulsion is miscible with water and may be added to a food, beverage, or supplement to provide a finished product. Products for oral administration include food products, food supplements, beverages, dietary supplements, and the like. For example, in an embodiment, the product for oral administration may comprise a solid, liquid, or semi-solid food. Non-limiting examples of solid food items include cookies, bars, brownies, and pancakes. According to a further aspect, the present invention provides a confectionary product such as chocolate. Cocoa processing is improved by lecithins, which allows for reduced cocoa butter input and improved chocolate quality. Lecithins in chocolate provide increased temperature resistance, extended shelf life and the preservation of appealing surfaces of chocolate products. Another solid food item is bakery products, which allows for even mixing of batter, decreased dough stickiness and reduced fat content. Lecithins allow for increased moisture retention in the final product and improved freshness and taste, and can increase volume yield, provide finer pores and better crusts in breads. In margarines and fats, lecithin provides help in avoiding spattering of hot fat and scorching of milk proteins in frying. Additionally, lecithin addition to chewing gum helps keep gum soft and elastic during chewing. In instant products, lecithin improves the dispersability of powders with high fat contents and increases the wettability of high protein ingredients. In particular, products include bar products, confectionary products which includes, e.g., jelly candies (gummies), soft candies, hard candies, chocolates and gums. Non-limiting examples of liquid foods include beers, malted beverages, non-alcoholic beers and malted beverages, shakes, juices, water, flavored water, teas, and coffees. In an embodiment, a semi-solid food item comprises between 10% and 90% liquid.

In other embodiments, the product for oral administration comprises additional components, such as one or more nutritive sweetener, bulking agent, or non-nutritive sweeteners. In these embodiments, the nutritive sweetener may be selected from the group consisting of sucrose, glucose, glucose syrup, isoglucose, fructose, glucose-fructose syrup, maltose, lactose, corn syrup, high fructose corn syrup, invert sugar, molasses, honey, agave and mixtures thereof; bulking agents which can include maltodextrin, polydextrose, xanthan gum, guar gum, soluble corn fiber (SCF), polyols, and mixtures thereof; non-nutritive sweeteners which can include high intensity sweeteners and sugar alcohols, mogroside, mogroside mixtures, aspartame, acesulfame-k, sucralose, steviol glycoside mixtures, stevia plant parts, and combinations thereof.

The product for oral administration includes food products, beverage products, and dietary supplements which are given orally or used in the oral cavity. Dietary supplements include vitamins, cough syrups, cough drops, chewable medicine tablets, amino acids, bitter-tasting agents, acidulants or the like), in a form such as a pill, tablet, spray, capsule, syrup, drop, troche agent, powder, and the like; personal care products such as other oral compositions used in the oral cavity such as mouth freshening agents, gargling agents, mouth rinsing agents, toothpaste, tooth polish, dentrifices, mouth sprays, teeth-whitening agent and the like. The present invention also includes tinctures and other products that are meant for sublingual administration.

The following examples are provided for illustrative purposes only and are not intended to limit the scope of the invention.

### EXAMPLES

### Example 1

A 4 L flask filled with 1.5 L of 8 g/L organic potato starch and 0.8 g/L organic carrot powder in RO water was sterilized and inoculated from a two week old P1 C. sinensis culture. After culturing for 7 days at room temperature at 60 RPM (1″ swing radius), the culture was filtered through three stacked coffee filters, pasteurized for 40 minutes at 165° F. and placed in a small batch desiccator at 140° F. overnight. The following day the dried material was collected and blended with a yield of 4.5 g/L for a total of 6.75 g. 5 g of the harvested material was poured into 1 L of RO water and shaken intermittently for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm.

### Example 2

A 4 L flask filled with 1.5 L of 8 g/L organic potato starch and 0.8 g/L organic carrot powder in RO water was sterilized and inoculated from a two week old P1 C. sinensis culture. After culturing for 15 days at room temperature at 60 RPM (1″ swing radius), the

In one embodiment, the present inventors have found that the a portion of a fungal aqueous culture fluid, the extracellular portion (containing reduced amounts of mycelium, herein also referred to as the “mycelium-free portion”) when added directly a composition for oral administration comprising a material such as, for example, a surfactant or an oil, has the ability to improve undesirable tastes in the composition, that are due to the surfactant or the oil in the composition. Such undesirable tastes include bitter tastes, grassy tastes, and/or other undesirable surfactant or oil-related flavors. Flavor/taste improvement also includes reduction of characteristic tastes and/or aftertastes associated with a surfactant or an oil, including, without limitation, a bitter flavor, a grassy flavor, an herbal flavor, a barnyard flavor.

Accordingly, the present invention relates to a composition for oral administration, wherein the composition comprises a combination of an extracellular portion from a mycelial aqueous culture comprising a filamentous fungus and a material comprising a surfactant or an oil, and wherein the composition has a reduced undesirable taste due to the surfactant or the oil in the composition, as well as methods by which to reducing undesirable tastes of a material comprising a surfactant or an oil in a composition for oral administration, by adding an extracellular portion from a mycelial aqueous culture comprising a filamentous fungus to the material comprising a surfactant or an oil in an amount sufficient to reduce at least one undesirable taste due to the surfactant or the oil in the composition for oral administration. The compositions comprising the combinations have reduced undesirable taste or tastes relative to the products for oral administration alone. The compositions optionally comprise an oil or a surfactant in an emulsified format.

Specifically, the inventors used an extracellular portion of a C. sinensis aqueous culture to mix with an oil or surfactant-containing composition, for the reason that the C. sinensis extracellular portion of an aqueous culture has taste improving and/or bitter blocker properties. The extracellular portion may be further purified, for example, to increase solubility, and may be dried, such as spray-drying, and combined with products for oral administration to improve the food products' taste profiles, including reducing bitter tastes and/or aftertastes. The present invention thus discloses a bitter blocker (the extracellular portion) that is effective for reducing undesirable flavors in oil or surfactant-containing compositions for oral administration.

In one embodiment, the present invention includes a method for reducing undesirable tastes of a material comprising a surfactant or an oil (or combinations thereof), which includes the steps of culturing a mycelial aqueous culture in an aqueous media, collecting an extracellular portion of the culture, and adding the extracellular portion to the material to reduce its undesirable tastes. In an embodiment, the material is part of a composition or product for oral administration. In another embodiment, the methods include the step of adding an extracellular portion of a mycelial aqueous culture to a composition comprising a surfactant or an oil in an amount effective to reduce at least one undesirable taste of the composition.

Accordingly, the present invention includes a method to make a composition for oral administration with at least one reduced undesirable taste, or a method to reduce an undesirable taste of a material comprising a surfactant or an oil in a composition for oral administration, comprising culturing a mycelial aqueous culture in a media, collecting the extracellular portion, and combining the extracellular portion of the culture and mixing it with the material or composition comprising an oil or surfactant. Appropriate fungi to use, appropriate media, appropriate methods of collecting the mycelium free portion of the extracellular portion are disclosed herein. The extracellular portion may be optionally concentrated, diluted or dried as disclosed herein, and may be combined with any food product as disclosed herein prior to use. The present invention also includes combination products comprising one or more food product(s) and an extracellular portion made from a mycelial aqueous culture made by the processes disclosed herein.

The lecithin for use with the present invention includes an edible lecithin from any number of sources, but commercially the most common lecithins are soy lecithin, egg yolk lecithin, and sunflower lecithin. Lecithins may be further processed or treated prior to use. For example, lecithin is available in liquid or powder (spray dried) forms. Lecithins may be treated by filtration, chemical or enzymatic modification and de-oiling to produce purified or modified lecithins. The molecular structure and polar head groups may be modified by either enzymatic or chemical means.

Soybean lecithins have long been known as food additives with excellent technological properties. They are used in various products as emulsifiers, stabilizers or dispersing agents in e.g. chocolate, instant products, margarine, mayonnaise, bakery products, and ice cream. Modified soybean lecithins and especially lysolecithins, treated with phospholipase A2, widen the field of applicability because of their increased polarity. However, these hydrolyzed lecithins also produce an off-flavor in the final products, which limits their use. The unpleasant off-flavor consists of an off-odor described mainly as straw-y, roasty, nutty, or haylike, and an off-taste described, above all, as bitter. To date little is known about the bitter-tasting compounds and their concentrations in commercial crude, standardized, and modified lecithins. One source of bitterness in soybean lecithin is polyunsaturated free linoleic acid (cis-9, cis-12-octadecadienoic acid) and free linolenic acid (cis-9, cis-12, cis-15-octadecatrienoic acid).

Oils to use with the invention include edible oils that have undesirable tastes, such as bitter tastes. Oils include, without limitation, any edible oil from any plant, fungal, or animal source. In some embodiments, the oils include olive oil, coconut oil, corn oil, cottonseed oil, palm oil, peanut oil, rapeseed/canola oil, safflower oil, sesame oil, soybean oil, sunflower oil, and/or combinations thereof. Oils of the invention also include nut oils, such as almond oil, beech nut oil, brazil nut oil, cashew oil, hazelnut oil, macadamia nut oil, mongongo nut oil, pecan oil, pine nut oil, pistachio oil, walnut oil, pumpkin see oil or citrus oils, such as lemon oil, orange oil, grapefruit seed oil. Other oils include the “nutraceutical” type oils such as borage seed oil, blackcurrant seed oil, evening primrose oil, acai oil, black seed oil, flaxseed oil, amaranth oil, apricot oil, apple seed oil, argan oil, avocado oil, babassu oil, ben oil, borneo tallow nut oil, cape chestnut oil, carob pod oil, cocoa butter, cohune oil, coriander seed oil, date seed oil, grape see oil, hemp oil, and the like.

Oils are highly lipophilic, and are best mixed with lipids or fats instead of water. However, oils and fats are difficult to intermix with materials such as foods or beverages, which are water-based. Therefore, in one embodiment, the oils of the invention are first prepared as an emulsion using a surfactant using known techniques, which is then diluted to form the composition for oral administration.

Oils are not soluble in water, but they show a good solubility in non-polar solvents such as oils and fats. It requires a suitable emulsification technique to overcome the immiscibility of oils in water. In embodiments, an oil emulsion can be prepared as an oil-in-water emulsion or water-in-oil emulsion using known art techniques; any of the below emulsion techniques can be used with the present invention.

Oil-in-water emulsions are visually homogeneous mixtures that combine the properties of both oil and water, wherein water is the continuous phase and oil is the dispersed phase, stabilized by at least one surfactant (emulsifier). There are three main types of oil-in-water emulsions: macroemulsions, microemulsions and nanoemulsions. Any of these types of emulsions are suitable to use with the present invention.

In one embodiment, the extracellular portion can be added to the composition for oral administration as part of an emulsion. The extracellular portion of the invention can be added to the emulsion at any number of different steps. One of skill in the art may determine the optimum method for incorporating the extracellular portion for maximum efficacy in a given application. In one embodiment, the extracellular portion is added to the water or aqueous portion of the emulsion prior to mixing with one or more other components of the emulsion. The extracellular component may be added to the water portion of the emulsion prior to incorporating into the other components of the emulsion. In order to obtain dissolution/suspension of the extracellular component, optionally, after addition, the aqueous portion is heated and/or sonicated, for example. Any method known in the art capable of enhancing dissolution/suspension may be used.

Optionally, the emulsifying agent may be added to the water portion before, after, or together with the extracellular component.

After the addition of the extracellular portion and/or emulsifying agent, the aqueous portion is then mixed with the optional oil portion of the emulsion. If the emulsifying agent was not added at a prior step, it may be added to an oil portion before, after or during the aqueous portion is mixed with the oil portion. The result is an emulsion that comprises the extracellular portion, oil, and emulsifying agent. In an embodiment, a source of energy is applied to form the emulsion. In one embodiment, the emulsion is a nanoemulsion and is formed by methods known in the art for forming a nanoemulsion, including ultrasonication. Methods for forming a nanoemulsion are known in the art.

Alternatively, the surfactant may be admixed with the extracellular portion in the absence of an oil and then blended with the oil to form the emulsion. culture was filtered through three stacked coffee filters, pasteurized for 40 minutes at 165° F. and placed in a small batch desiccator at 140° F. overnight. The following day the dried material was collected and blended with a yield of 4.1 g/L for a total of 6.15 g. 5 g of the harvested material was poured into 1 L of RO water and shaken intermittently for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm.

### Example 3

A 4 L flask filled with 1.5 L of 8 g/L organic potato starch and 0.8 g/L organic carrot powder in RO water was sterilized and inoculated from a two week old P1 C. sinensis culture. After culturing for 35 days at room temperature at 60 RPM (1″ swing radius), the culture was filtered through three stacked coffee filters, pasteurized for 50 minutes at 165° F. and placed in a small batch desiccator at 140° F. overnight. The following day the dried material was collected and blended with a yield of 5.5 g/L for a total of 8.25 g. 5 g of the harvested material was poured into 1 L of RO water and shaken intermittently and heated on a hot plate turned to medium for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm.

### Example 4

A 4 L flask filled with 1.5 L of 8 g/L organic potato starch and 0.8 g/L organic carrot powder in RO water was sterilized and inoculated from a two week old P1 C. sinensis culture. After culturing for 7 days at room temperature at 60 RPM (1″ swing radius), the culture was filtered through cheesecloth, pasteurized for 50 minutes at 160° F. and placed in a small batch desiccator at 130° F. overnight. The following day the dried material was collected and blended with a yield of 4.4 g/L for a total of 6.6 g. 5 g of the harvested material was poured into 1 L of RO water and shaken intermittently for 15 minutes. From this stock culture, 53.34 mL of solution was added to another solution containing 1 kg of 97% rebaudioside A dissolved in 1.6 L of RO water. This solution was thoroughly mixed and dried in a small batch desiccator overnight, and the resulting material was blended and packaged in a clean ziplock bag, having a concentration of the collected filtrate solids of 2,667 ppm.

### Example 5

A 4 L flask filled with 1.5 L of 8 g/L organic potato starch and 0.8 g/L organic carrot powder in RO water was sterilized and inoculated from a two week old P1 C. sinensis culture. After culturing for 10 days at room temperature at 60 RPM (1″ swing radius), the culture was filtered through three stacked coffee filters, pasteurized for 40 minutes at 170° F. and placed in a small batch desiccator at 140° F. overnight. The following day the dried material was collected and blended with a yield of 4.6 g/L for a total of 6.9 g. 5 g of the harvested material was poured into 1 L of RO water and shaken intermittently for 15 minutes. From this stock culture, 40.00 mL of solution was added to another 1.6 L solution of distilled water containing 1 kg of 97% rebaudioside A. This solution was thoroughly mixed and dried in a small batch desiccator overnight, and the resulting material was blended and packaged in a clean ziplock bag, having a concentration of the collected filtrate solids of 2,000 ppm.

### Example 6

The C. sinensis extracellular portion powder (bitter blocker, produced by the methods outlined in Example 1-5) was used.

An emulsion was formed by mixing MCT (medium chain triglyceride) oil, ethylcellulose (Ethocel) N100 (Dow Chemical) and bitter blocker of the present invention.

A 0.5% (w/w) dispersion of ethyl cellulose N100 (Dow) in MCT oil was made by weighing the two components (e.g. 0.5 g ethyl cellulose plus 99.5 g MCT oil) and mixing gently to disperse the ethyl cellulose. Ethyl cellulose dispersed easily into the oil resulting in a turbid mixture with particles. Ethyl cellulose did not dissolve in MCT oil at room temperature.

In a Pyrex beaker, the mixture was heated to 146° C.±1° C. while stirring on a heated magnetic stir plate and mixed well. Stirring was vigorous such that the ethyl cellulose particles remained homogeneously dispersed but not so much that a full vortex was created. Once heated to 146° C., this temperature was maintained for about 3 minutes or until the ethyl cellulose was completely dissolved.

The solution was allowed to cool slowly over an approximate 30-minute period while stirring. At room temperature, the product was slightly cloudy/translucent. At that time, extracellular portion was added to create the desired final concentration dispersion (e.g., 10 mg added to 100 mL MCT-ethyl cellulose solution created a 100 ppm dispersion. Mixtures were made with extracellular portion to 300 ppm, or 500 ppm dispersion. The mixture was mixed to disperse the extracellular portion into the solution. The solution was slightly turbid.

The extracellular portion dispersion was then mixed with CBD:THC 3:1, CBD, THC, and “full spectrum” hemp oil at various concentrations of between 5 and 30 mg/mL. The extracellular portion in the mixture was present at a final concentration of 40 to 300 ppm and the mixture was tasted, and compared with a similar mixture without extracellular portion. The tasters agreed that mixtures with extracellular portion were significantly less bitter and had less grassy/cannabinoid taste than mixtures without extracellular portion. The mixtures were diluted into a “tea” type drink at 1 ml mixture to about 200 to 500 mL of tea. Similar results as to the mixtures alone for the tasting were obtained.

### Example 7

The C. sinensis extracellular portion powder (bitter blocker, produced by the methods outlined in Example 1-5) was used.

To create the nanoemulsion.

Active ingredient (e.g., cannabis oil extract), 5.40%

Carrier Oil (e.g. olive oil), 7.20%.

Surfactant (e.g., soy lecithin) 2.00%

Water 85.4% (containing 100 ppm extracellular portion)

Ultrasonification was carried out using a Hielscher Ultrasonic Technology UP400St, where the sonotrode is placed into the mixture, set to 50% amplitude. The oil mix was slowly poured into the water solution while sonicating the water to form a nanoemulsion. To achieve a better nanoemulsion, the oil mix was added into the water right under the sonotrode using an eyedropper or syringe. It was found that ultrasonic cavitation was a very effective emulsification method to prepare superior emulsions in the nano range. The nanoemulsion was tasted, and compared with a similar mixture without extracellular portion and tasters agreed that mixtures with the extracellular portion were significantly less bitter and had less grassy/cannabinoid taste than mixtures without extracellular portion. The mixtures were diluted into a “tea” type drink at 1 ml mixture to about 200 to 500 mL of tea. Similar results as to the mixtures alone for the tasting were obtained.

### Example 8

The C. sinensis extracellular portion powder (bitter blocker, produced by methods outlined in Example 1-5) is used.

The extracellular portion powder was diluted into liquid lecithin and mixed via shaking. The extracellular portion was observed to disperse into the lecithin but was not completely solubilized. The lecithin/extracellular mixture was then mixed with canola oil. The mixture was gently heated, resulting in a clear mixture. The mixture is tasted and found to have a milder, less bitter taste compared to an oil control (lecithin lacking extracellular portion). The lecithin/extracellular mixtures are then mixed into chocolate. The mixture is tasted and the chocolate is found to have a milder, less bitter taste than the control.

The extracellular portion powder is diluted into a small amount of water, between 10 ppm and 10,000 ppm final concentration, then is mixed with liquid lecithin to dissolve the extracellular portion. In another experiment, the extracellular portion is diluted into a small amount of water, between 10 ppm and 10,000 ppm final concentration, and then is mixed with dry or powdered lecithin. The mixture is tasted and found to have a milder, less bitter taste compared to a lecithin control (lecithin lacking extracellular portion). Foods/beverages incorporating the lecithin/extracellular portion mixtures also have a milder, less bitter taste.

The description of the various embodiments has been presented for purposes of illustration and description but is not intended to be exhaustive or limiting of the invention to the form disclosed. The scope of the present invention is limited only by the scope of the following claims. Many modifications and variations will be apparent to those of ordinary skill in the art. The embodiments described were chosen and described in order to explain the principles of the invention, the practical application, and to enable others of ordinary skill in the art to understand the invention for various embodiments with various modifications as are suited to the particular use contemplated. All references cited herein are incorporated in their entirety by reference.

1. A composition for oral administration, wherein the composition comprises a combination of an extracellular portion from a mycelial aqueous culture comprising a filamentous fungus, and at least one of a cannabis oil or a cannabinoid and optionally, a material comprising a surfactant or an oil, and wherein the composition has a reduced undesirable taste compared with the same composition lacking the extracellular portion.

2. The composition of claim 1, wherein the filamentous fungus is selected from the group consisting of: Ganoderma lucidum, Ganoderma applanatum, Cordyceps sinensis, Cordyceps militaris, Hericium erinaceus, Lentinula edodes, Agaricus blazei, Grifola frondosa, Auricularia auricula, Flammulina velutipes, Trametes versicolor, Morchella spp., Inonotus obliquus, Laricifomes officinalis, Fomes fomentarius, Fomes officinalis, Fomes fomitopsis, Tricholoma matsutake, Boletus edulis, Clitocybe nuda, Clitocybe saeva, Plearotus spp., Tremella fuciformis, Piptoporus betulinus, Polyporus umbellatus, Pholiota nameko, Volvariella volvacea, Hypsizygus marmoreus, Stropharia rugosoannulata, and Laetiporus sulphureus.

3. The composition of claim 2, wherein the filamentous fungus is Cordyceps sinensis.

4. The composition of claim 1, wherein the extracellular portion of the mycelial aqueous culture is obtained by filtration or centrifugation of the mycelial aqueous culture.

5. The composition of claim 1, wherein the extracellular portion of the mycelial aqueous culture is prepared by a method comprising:
culturing the mycelial aqueous culture in a media;
separating the extracellular portion from the mycelial cells; and
collecting the extracellular portion of the mycelial aqueous culture.

6. The composition of claim 1, wherein the reduced undesirable taste comprises at least one of reduced bitter tastes, reduced undesirable aftertastes, and/or reduced astringency compared to the product for oral administration alone.

7. The composition of claim 1, wherein the extracellular portion from the mycelial aqueous culture is pasteurized or sterilized.

8. The composition of claim 1, wherein the extracellular portion from the mycelial aqueous culture is collected by filtration or centrifugation.

9. The composition of claim 1, wherein the product for oral administration is a beverage, a food, or a dietary supplement.

10. The composition of claim 1, wherein the cannabis oil or cannabinoid is incorporated into an oil-in-water or a water-in-oil emulsion.

11. The composition of claim 10, wherein the extracellular portion is incorporated into the oil-in-water or a water-in-oil emulsion.

12. The composition of claim 1, wherein the composition comprises a surfactant and the surfactant is lecithin.

13. The composition of claim 12, wherein the lecithin comprises soy lecithin, sunflower lecithin, or egg lecithin.

14. The composition of claim 13, wherein the surfactant is soy lecithin.

15. The composition of claim 1, wherein composition comprises an oil and the oil comprises medium chain triglycerides.

16. The method of claim 10, wherein the emulsion is incorporated into the product for oral administration.

17. A method for reducing undesirable tastes in a composition comprising a cannabinoid or cannabis oil for oral administration, comprising:
adding an extracellular portion from a mycelial aqueous culture comprising a filamentous fungus to the cannabinoid-containing or cannabis oil-containing product for oral administration in an amount sufficient to reduce at least one undesirable taste in the composition for oral administration.

18. The method according to claim 17, wherein the undesirable taste comprises at least one of bitter taste or undesirable aftertaste.

19. The method according to claim 17, wherein the cannabinoid or cannabis oil containing product is present in the composition for oral administration in the form of an emulsion.

20. A composition for oral administration, wherein the composition comprises a combination of an extracellular portion from a mycelial aqueous culture comprising a filamentous fungus and a material comprising a surfactant or an oil, and wherein the composition has a reduced undesirable taste from the surfactant or the oil in the composition.

21. The composition of claim 20, wherein the filamentous fungus is selected from the group consisting of: Ganoderma lucidum, Ganoderma applanatum, Cordyceps sinensis, Cordyceps militaris, Hericium erinaceus, Lentinula edodes, Agaricus blazei, Grifola frondosa, Auricularia auricula, Flammulina velutipes, Trametes versicolor, Morchella spp., Inonotus obliquus, Laricifomes officinalis, Fomes fomentarius, Fomes officinalis, Fomes fomitopsis, Tricholoma matsutake, Boletus edulis, Clitocybe nuda, Clitocybe saeva, Plearotus spp., Tremella fuciformis, Piptoporus betulinus, Polyporus umbellatus, Pholiota nameko, Volvariella volvacea, Hypsizygus marmoreus, Stropharia rugosoannulata, and Laetiporus sulphureus.

22. The composition of claim 21, wherein the filamentous fungus is Cordyceps sinensis.

23. The composition of claim 20, wherein the extracellular portion of the mycelial aqueous culture is obtained by filtration or centrifugation of the mycelial aqueous culture.

24. The composition of claim 20, wherein the extracellular portion of the mycelial aqueous culture is prepared by a method comprising:
culturing the mycelial aqueous culture in a media;
separating the extracellular portion from the mycelial cells; and
collecting the extracellular portion of the mycelial aqueous culture.

25. The composition of claim 20, wherein the reduced undesirable taste comprises at least one of reduced bitter tastes, reduced undesirable aftertastes, and/or reduced astringency compared to the product for oral administration alone.

26. The composition of claim 20, wherein the extracellular portion from the mycelial aqueous culture is collected by filtration or centrifugation.

27. The composition of claim 20, wherein the product for oral administration is a beverage, a food, or a dietary supplement.

28. The composition of claim 20, wherein the oil or surfactant is incorporated into an oil-in-water or a water-in-oil emulsion.

29. The composition of claim 28, wherein the extracellular portion is incorporated into the oil-in-water or a water-in-oil emulsion.

30. The composition of claim 20, wherein the surfactant is lecithin.

31. The composition of claim 20, wherein the oil is olive oil.

32. The method of claim 28, wherein the emulsion is incorporated into the product for oral administration.

33. A method for reducing undesirable tastes of a material comprising a surfactant or an oil in a composition for oral administration, comprising:
adding an extracellular portion from a mycelial aqueous culture comprising a filamentous fungus to the material comprising a surfactant or an oil in an amount sufficient to reduce at least one undesirable taste from the surfactant or the oil in the composition for oral administration.

34. The method according to claim 33, wherein the undesirable taste comprises at least one of bitter taste or undesirable aftertaste.

35. The method according to claim 34, wherein the oil or surfactant is present in the composition for oral administration in the form of an emulsion.
